(19) United States
(12) Patent Application Publication
(10) Pub. No.: US 2025/0366873 A1
(43) Pub. Date: Dec. 4, 2025

(54) MEDICAL DEVICES AND RELATED SYSTEMS AND METHODS OF USE

(71) Applicant: Boston Scientific Scimed, Inc., Maple Grove, MN (US)

(72) Inventors: Michael S. H. CHU, Brookline, MA (US); Gene Thomas STORBECK, Millis, MA (US); Niraj Prasad RAUNIYAR, Plymouth, MN (US)

(73) Assignee: Boston Scientific Scimed, Inc., Maple Grove, MN (US)

(21) Appl. No.: 19/216,928

(22) Filed: May 23, 2025

Related U.S. Application Data
(60) Provisional application No. 63/652,910, filed on May 29, 2024.

(51) Int. Cl. A61B 17/22; A61B 17/00

(52) U.S. Cl. CPC A61B 17/22; A61B 2017/00367; A61B 2017/0042; A61B 2017/22079

(57) ABSTRACT

In some aspects, this disclosure includes a medical device comprising: a sheath extending from a proximal end to a distal end. The sheath includes a first lumen extending from the proximal end to the distal end. The medical device further comprises a proximal body coupled to the proximal end of the sheath and a second lumen extending longitudinally through the proximal body; and a first body extending radially outward from the proximal body, and the first body includes a third lumen extending longitudinally through the first body. The first lumen, the second lumen, and the third lumen are fluidically connected. The medical device further comprises a seal positioned at a proximal portion of the proximal body; and an actuator. The actuation of the actuator adjusts a size of a vent of the medical device.

### CROSS-REFERENCE TO RELATED APPLICATIONS

This application claims the benefit of priority from U.S. Provisional Application No. 63/652,910, filed on May 29, 2024, which is incorporated by reference herein in its entirety.

### TECHNICAL FIELD

Various aspects of this disclosure relate generally to medical devices, systems, and procedures. Particular aspects relate to medical devices, systems, and procedures for removing material, e.g., obstructions, from one or more body lumens and/or passageways, among other aspects.

### BACKGROUND

Medical devices, such as lasers, needles, retrieval devices, infusion tubes, sensors, and the like may include an elongate body, and may be arranged for delivery through a working channel of an insertion device (e.g., a ureteroscope, a hysteroscope, a bronchoscope, a cystoscope, endoscope, sheath, and similar devices). The elongate body of such medical devices may be selectively extended and/or retracted relative to the working channel of the insertion device to deploy or retract the elongate body to perform one or more therapies, treatments, or diagnostic evaluations on a subject. For example, the medical device may include a laser having an elongate body arranged for delivery through a working channel of an ureteroscope. Such elongated medical devices often must be threaded through a seal or valve in order to help prevent irrigation or fluid backflow through the port or channel through which the elongated device accesses the insertion device. An oversheath may be utilized to accommodate the ureteroscope and the elongated medical device, and may supply a vacuum source while aspiration is applied to the ureteroscope. However, there is the danger of over-pressurizing a body lumen of a patient, and potentially injuring the patient as a result. Such injuries can increase the cost, complexity, and/or risks of the medical procedure involving an insertion device.

Furthermore, the medical devices may be used to apply both irrigation and suction (negative pressure) to remove renal stones or fragments of renal stones (or other material). As a result, the interior of the patient's body may become over-pressurized with saline without the proper measures to provide drainage and/or adjustment to the applied suction and irrigation.

The devices, systems, and methods of this disclosure may rectify some of the deficiencies described above or address other aspects of the prior art.

### SUMMARY

This disclosure relates to, among other things, medical devices and systems for removing obstructions from body lumens and/or passageways, and related methods of use. Aspects of this disclosure may help to decrease the time to remove an obstruction, such as a kidney stone, from a patient's body. Further, for example, aspects of this disclosure may help to reduce the level of skill of a practitioner sufficient to complete such obstruction removal procedure, and/or may help to prevent damage to one or more devices during a procedure. Although primarily discussed in the context of kidney stone removal procedures, any of the systems, devices, and features thereof discussed herein may be used or adapted for use in other parts of the body to remove other materials or obstructions, or be used in other manners besides material removal. The medical devices and systems disclosed herein in various examples may include one or more of the features or components described in connection with any of the other examples.

In some aspects, this disclosure includes a medical device comprising: a sheath extending from a proximal end to a distal end. The sheath includes a first lumen extending from the proximal end to the distal end. The medical device further comprises a proximal body coupled to the proximal end of the sheath and including a second lumen extending longitudinally through the proximal body; and a first body extending radially outward from the proximal body, and the first body includes a third lumen extending longitudinally through the first body. The first lumen, the second lumen, and the third lumen are fluidically connected. The medical device further comprises a seal positioned at a proximal portion of the proximal body; and an actuator. The actuation of the actuator is configured to adjust a size of a vent of the medical device.

In other aspects, the medical device may include one or more of the following features. The first body may be a handle extending from the proximal body, wherein a longitudinal axis of the handle forms a non-perpendicular angle with a longitudinal axis of the proximal body; and wherein the handle is configured to be coupled to a suction source. Each of the actuator and the vent may be positioned on the handle. A dilator may be positioned within the sheath and/or the proximal body. The actuator may be C-shaped and may include an elastic member. At least one protrusion of the actuator may be received within a track of the proximal body or the first body. The track may include a series of recesses configured to receive the at least one protrusion of the actuator. A longitudinal axis of the track may not be parallel with a central longitudinal axis of the first body. A biasing member may be positioned within the sheath and the proximal body. The biasing member may be coupled to a ring member at a proximalmost end of the biasing member. The biasing member may be coupled to a sleeve, and the sleeve may be positioned within the second lumen. The first body may be a curved arm extending from a first side of the proximal body, and the actuator may be positioned on a second side of the proximal body, and the first side may be opposite from the second side. The first body may be a curved arm extending from a first side of the proximal body, and the actuator may be positioned on a handle extending from a second side of the proximal body, and the first side may be opposite from the second side. The actuator may be rotatable around a circumference of the second body. Proximal body may include a pair of arms extending radially outward from a flange of proximal body, and each arm of the pair of arms may include an opening configured to receive a portion of a dilator hub.

In other aspects, a medical device may comprise: a sheath extending from a proximal end to a distal end, wherein the sheath includes a first lumen extending from the proximal end to the distal end; a proximal body coupled to the proximal end of the sheath and including a second lumen extending longitudinally through the proximal body, wherein the first lumen and the second lumen are fluidically connected; and an actuator, wherein actuation of the actuator is configured to adjust a size of a vent of the medical device.

In other aspects, the medical device may include one or more of the following features. A seal at a proximal end of the proximal body, and the seal includes a plurality of lumens. A seal cap coupled to a proximal end of the proximal body, and the seal cap includes a pair of protrusions configured to couple a dilator hub to the seal cap.

In other aspects, a medical device may comprise a sheath extending from a proximal end to a distal end, wherein the sheath includes a first lumen extending from the proximal end to the distal end; a proximal body coupled to the proximal end of the sheath and including a second lumen extending longitudinally through the proximal body, wherein the first lumen and the second lumen are fluidically connected, and wherein the proximal body include a vent; an actuator, wherein actuation of the actuator is configured to transition from (i) a first position completely covering the vent to (ii) a second position completely not covering from the vent; and a seal coupled to the proximal body and configured to receive an insertion device.

In other aspects, the medical device may include one or more of the following features. A dilator including a dilator hub, and the dilator hub may include a pair of arms extending outward from a central body of the dilator; and a seal cap coupled to the proximal body. The seal cap may include a pair of protrusions configured to engage the pair of arms of the dilator hub to couple the dilator hub to the seal cap.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, device, or apparatus that comprises a list of elements does not include only those elements, but may include other elements not expressly listed or inherent to such process, method, article, device, or apparatus. The term “exemplary” is used in the sense of “example,” rather than “ideal.” The term “distal” refers to a portion farthest away from a user when introducing a device into a patient. By contrast, the term “proximal” refers to a portion closest to the user when placing the device into the patient. Proximal and distal directions are labeled with arrows marked “P” and “D”, respectively, throughout various figures. Although ureteroscopes are referenced herein, reference to ureteroscopes should not be construed as limiting the possible applications of the disclosed aspects. For example, the disclosed aspects may be used with endoscopes, duodenoscopes, bronchoscopes, colonoscopes, catheters, sheaths, diagnostic or therapeutic tools or devices, or other types of medical devices. Further, relative terms such as, for example, “about,” “substantially,” “approximately,” etc., are used to indicate a possible variation of ±10% in a stated numeric value or range.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIG. 1 is a side view of an exemplary medical device, according to aspects of this disclosure.

FIGS. 2A-2C illustrate portions of the medical device of FIG. 1, according to aspects of this disclosure.

FIG. 3 illustrates an exemplary medical system including the medical device of FIG. 1, respectively, according to aspects of this disclosure.

FIGS. 4A and 4B illustrate components of the medical device of FIG. 1, according to aspects of this disclosure.

FIGS. 5 and 6 illustrate embodiments of a component of a medical device, according to aspects of this disclosure.

FIG. 7 illustrates a relative position of a track of a medical device relative to a center line of a medical device, according to aspects of this disclosure.

FIG. 8 is a side view of an exemplary medical device, according to aspects of this disclosure.

FIGS. 9, 10, and 11 illustrate portions of the medical device of FIG. 8, according to aspects of this disclosure.

FIG. 12 is an exploded view of the exemplary medical device shown in FIG. 8, according to aspects of this disclosure.

FIGS. 13A and 13B illustrate portions of the medical device of FIG. 8, according to aspects of this disclosure.

FIG. 13C illustrates a front view of an exemplary component of a medical device, according to aspects of this disclosure.

FIG. 14 is a side view of an exemplary medical device, according to aspects of this disclosure.

FIG. 15 illustrates an exploded view of the medical device of FIG. 14, according to aspects of this disclosure.

FIG. 16 is a side view of an exemplary medical device, according to aspects of this disclosure.

FIG. 17 illustrates an exploded view of the medical device of FIG. 16, according to aspects of this disclosure.

FIG. 18 illustrates a perspective view of an exemplary medical device, according to aspects of this disclosure.

FIG. 19 illustrates a magnified portion of the medical device of FIG. 18, according to aspects of this disclosure.

FIG. 20 illustrates an exploded view of the medical device of FIG. 18, according to aspects of this disclosure.

FIGS. 21 and 22 illustrate portions of the medical device of FIG. 18, according to aspects of this disclosure.

FIG. 23 illustrates a perspective view of an exemplary medical device, according to aspects of this disclosure.

FIG. 24 illustrates a magnified portion of the medical device of FIG. 23, according to aspects of this disclosure.

FIG. 25 illustrates a portion of the medical device of FIG. 23, according to aspects of this disclosure.

FIG. 26 illustrates a portion of the medical device of FIG. 23, according to aspects of this disclosure.

FIG. 27 illustrates a perspective view of an exemplary medical device, according to aspects of this disclosure.

FIG. 28 illustrates an exploded view of the exemplary medical device of FIG. 27, according to aspects of this disclosure.

FIGS. 29 and 30 illustrate top views of exemplary actuators, according to aspects of this disclosure.

FIG. 31 illustrates a proximal-facing view of the exemplary medical device of FIG. 27, according to aspects of this disclosure.

FIG. 32 illustrates a side view of exemplary actuator, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts.

Aspects of various devices are now described. Some aspects are described with reference to noninvasive procedures, such as urology procedures, wherein a sheath is advanced to a treatment site, and a capture device is extended from the sheath to engage an object at the treatment site. In urology procedures, for example, the sheath may be inserted into the urethra, moved through the bladder and ureter, and advanced into a calyx of a kidney; and the capture device may be extended distally from the sheath and/or a working channel of an ureteroscope or other device to engage one or more stones and/or stone fragments located in the calyx.

References to a particular type of procedure, such as a urology procedure; medical device, such as a laser, knife, basket or grasper; organ, such as a kidney; and/or object, such as a stone or stone fragment, are provided for convenience and not intended to limit this disclosure. Accordingly, the concepts described herein may be utilized for any analogous device or system.

Examples of this disclosure relate to medical device introducer sheaths for introduction of an elongate body of the medical device into a seal, valve, scope, or port of an insertion device. The introducer sheath may be used to assist in delivery of the elongate body into any appropriate insertion device. In addition, introducer sheaths described herein may provide a conduit to apply suction or fluids to a treatment site, among other aspects.

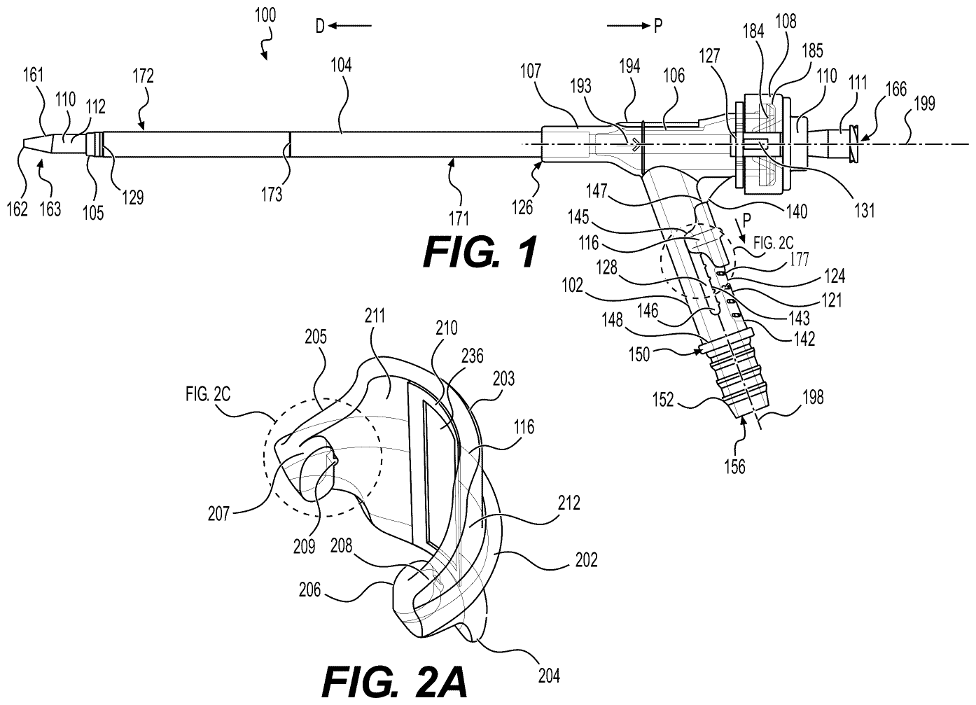

An exemplary medical device 100 is depicted in FIG. 1. Medical device 100 may be used to apply suction and/or aspirate a target area of a patient's body, and may be configured to receive one or more insertion devices 306 (FIG. 3). As shown in FIG. 1, device 100 may comprise: a handle 102; a sheath 104; a proximal body 106; and an actuator 116 movably mounted to handle 102. Sheath 104 may be coupled to a fitting 107 of proximal body 106, and may extend out of an opening 126 at a distalmost end of fitting 107. In some examples, fitting 107 may be removable from proximal body 106, and in other examples, fitting 107 may be integral with (and not removable from) proximal body 106. Handle 102 may extend radially outward, relative to a central longitudinal axis 199 of the sheath 104 and the proximal body 106, from the proximal body 106. Handle 102 may extend proximally from proximal body 106, and a central longitudinal axis 198 of handle 102 may form a non-perpendicular angle with central longitudinal axis 199. A seal 108 may be coupled to proximal body 106 at a proximalmost end of proximal body 106. In FIG. 1, medical device 100 includes a dilator 110. However, in other examples, medical device 100 may not include dilator 110.

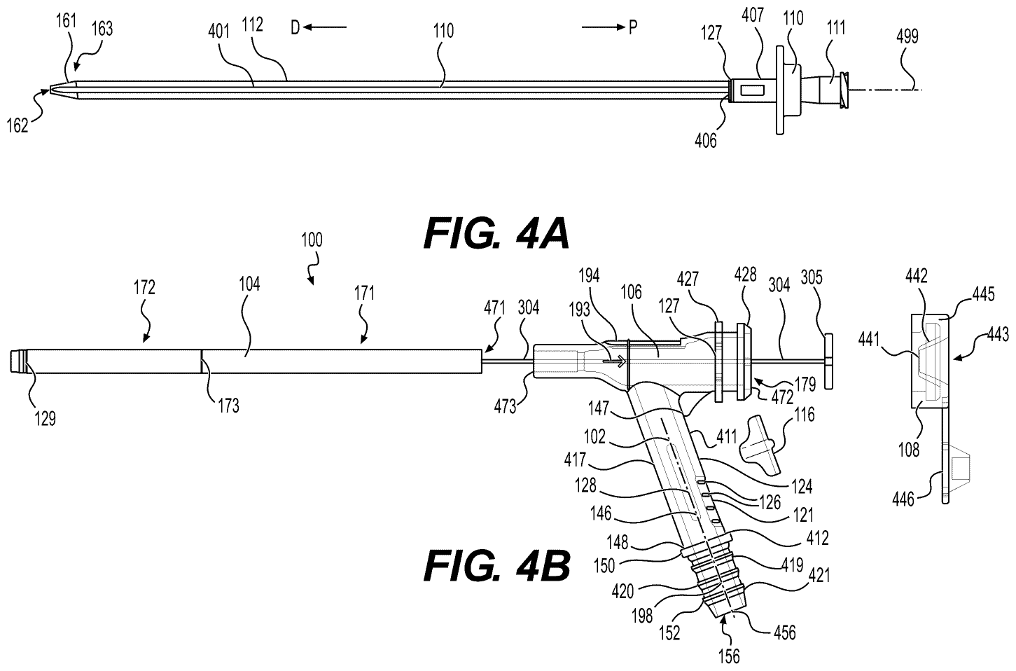

Dilator 110 may include a dilator hub 111, a shaft 112 extending distally from the dilator hub 111, and a distal end 163. Shaft 112 may be cylindrical and may be configured to be received within sheath 104. Dilator hub 111 may include one or more latches 127 configured to couple to a proximal end of proximal body 106, as shown in FIG. 1. Referring to FIG. 4A, each of the one or more latches 127 may be L-shaped (e.g., in a longitudinal cross-section), may include a first longitudinally extending portion 407 and a second portion 406 extending radially-inward towards shaft 112. Each of the one or more latches 127 may be configured to engage a flange 427 of proximal body 106 to couple dilator 110 to proximal body 106.

Distal end 163 of dilator 110 may include a tapered tip 161 and an opening 162 at the distalmost end of dilator 110. A longitudinally extending lumen 401 (shown in FIG. 4A) of dilator 110 may extend from opening 162 to opening 166 at a proximalmost end of dilator 110. Dilator 110 may be configured to be inserted into proximal body 106 and sheath 104, to facilitate with positioning medical device 100 at a target site within a patient's body and/or to facilitate dilating one or more of a patient's internal body lumens. Dilator 110 may be removed from medical device 100 by releasing the one or more latches 127, for example, by pushing/pulling each latch 127 radially outward, relative to longitudinal axis 199, and then pulling dilator hub 111 proximally dilator hub 111 proximally.

Sheath 104 may be cylindrical and may include a central lumen 471 (shown in FIG. 4B) extending longitudinally through sheath 104 from a proximalmost end of sheath 104 to a distalmost end 105 of sheath 104. Sheath 104 may include a flexible distal portion 172 and a relatively stiffer proximal portion 171, and flexible distal portion 172 may be configured to be articulated (e.g., bent in an up, down, left, and/or right direction) when an insertion device 306 is positioned within sheath 104 and is articulated in one or more directions. Sheath 104 may include one or more markings 129, 173 to facilitate visually identifying different portions of sheath 104. Marking 128 may be at a distal end of sheath 104, and marking 173 may be positioned along a length of sheath 104. Sheath 104 may be made of any suitable material, and in some examples, sheath 104 may be made of multiple layers of polymers and/or may be reinforced with a metal and/or plastic coil or braid. In some examples, distal portion 172 may be opaque or clear, or the entirety of sheath 104 may be opaque or clear. The outer surface of sheath 104 may be coated in a hydrophilic material.

In some examples, sheath 104 may be lined with a laser resistant material, may be either partially or fully lined, and the laser resistant material may coat the entirety of sheath 104 that forms lumen 471. A laser resistant material may be any material that can withstand high temperatures, such as the temperature of a laser beam used in a urological procedure, without melting, burning, or degrading. The laser resistant material may have a low thermal expansion, high thermal conductivity, high electrical conductivity, high corrosion resistance, and/or may be porous. In some examples, the laser resistant material may be clear or opaque, such that a user may at least partially see through the laser resistant material. By providing laser resistant material in sheath 104, a user may more safely laser stones to a smaller size within the inner diameter of sheath 104 (e.g., within lumen 471) without damaging sheath 104 or surrounding tissue. In medical device 100, a user may simultaneously laser material within sheath 104 and apply suction to sheath 104 to pull material out of sheath 104. Sheath 104 may be coupled to a fitting 194 of proximal body 106, and in some examples, may be glued to fitting 194 and/or insert molded to fitting 194.

Proximal body 106 may extend longitudinally along central longitudinal axis 199, and may include an interior lumen 179 extending from a proximal opening 472 to a distal opening 473 of proximal body 106. An indicator 193 may be on a side of proximal body 106, and indicator 193 may be the shape of an arrow and/or a line to indicate the insertion device 306 withdrawal limit into fitting 107 to aspirate material out of handle 102. For example, a user may desire to remove insertion device 306 from sheath 104 to allow passage of larger material through sheath 104 and out of handle 102, and thus may pull insertion device 306 proximally out of sheath 104 and proximally beyond indicator 193, to allow the material to move through sheath 104 and handle 102 without the insertion device 306 taking up space within sheath 104 and handle 102 (i.e., the passage of material through medical device 100). In some examples, as shown in FIG. 1, indicator 193 may have a proximalmost end that is aligned with a distalmost portion of handle 102.

Referring to FIG. 4B, proximal body 106 may include flange 427, which may extend radially outward, relative to central axis 199, and may be configured to abut a distalmost end of seal 108. Flange 427 may be configured to help prevent distal movement of seal 108 during operation of medical device 100. A tapered protrusion 428 may be at a proximalmost end of proximal body 106, and tapered protrusion 428 may be sized to receive seal 108, such that seal 108 fits around and/or couples to the tapered protrusion 428. A proximal portion of sheath 104 may extend within lumen 179 of proximal body 106.

Handle 102 may extend from proximal body 106, and a central lumen 158 may extend longitudinally through handle 102, be fluidically connected with lumen 179, and may extend to a proximal opening 456 at a proximal end 152 of handle 102. As shown in FIG. 4B, proximal end 152 of handle 102 may include a series of protrusions 419, 420, 421 protruding radially outward from the central longitudinal axis 198 of handle 102, and each protrusion 419-421 may be configured to facilitate coupling to a suction supply source, such as a tube connected to a vacuum source or other suction supply source. A flange 150 may extend circumferentially around a portion of handle 102 and may be configured to abut a tube or other member coupled to handle to supply a vacuum source to handle 102.

A track 128 may extend longitudinally along handle 102 and may form a recessed portion the radially-outer surface 417 of handle 102. In some examples, handle 102 may include two tracks 128 positioned on opposite sides of handle 102, and each track 128 may be configured to receive a portion of actuator 116. Track 128 may include a distal end 145 and a proximal end 146, and actuator 116 may be configured to move through track 128 between the distal end 145 and the proximal end 146. A vent 121 may extend longitudinally along handle 102, and vent 121 may fluidically connect a lumen 156 within handle 102 with an exterior of handle 102. Vent 121 may be positioned between two tracks 128, and may face a proximal direction (e.g., may be on a proximal side of handle 102. In other examples, vent 121 may face a distal direction, may face a lateral direction, or any other direction. One or more markings 177 may be positioned proximate to vent 121, and may facilitate positioning of actuator 116 relative to vent 121.

Actuator 116 may be moveably coupled to handle 102, and may be configured to slide longitudinally across at least a portion of handle 102. Actuator 116 may move from a first position abutting a first end 147 of handle 102 to a second position abutting a second end 148 of handle 102. In some examples, actuator 116 may entirely cover vent 121 when in the first position, and may be entirely spaced from vent 121 when in the second position. A user may adjust the position of actuator 116 along handle 102 to partially or fully cover vent 121. By adjusting the position of actuator 116 over vent 121, a user may control the magnitude of the suction force applied to sheath 104 through a suction source coupled to handle 102. For example, to increase the suction applied to sheath 104, a user may slide actuator 116 in a first direction towards vent 121 to partially or completely cover vent 121. To decrease the suction applied to sheath 104, a user may move actuator in a second direction opposite the first direction to partially or fully uncover vent 121. Actuator 116 may be adjusted when a different mode of aspiration (e.g., suction) is needed. In some examples, a user may slide actuator 116 back and forth to produce pulsation suction. In some examples, a user may push actuator 116 towards handle 102 to create a tighter seal with handle 102, to increase the magnitude of suction (momentarily) applied to sheath 104 through handle 102, or a user may move their finger to completely cover vent 121 momentarily to increase the suction force applied to sheath 104. In some examples, the size of one or more vents 121 may be small such that particulate/stones do not come out of the one or more vents 121.

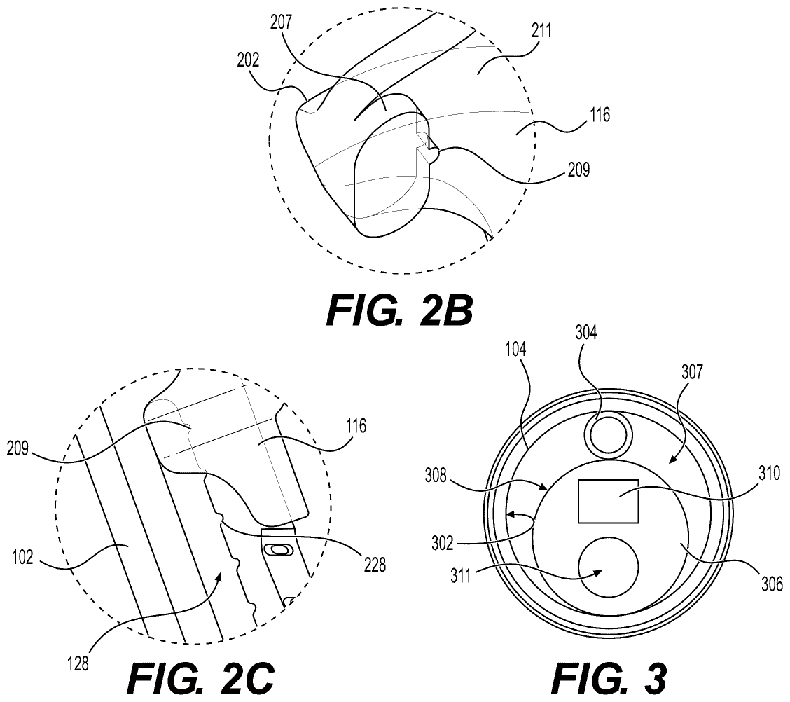

A magnified view of actuator 116 is shown in FIG. 2A. Actuator 116 may be U-shaped and may include a central body portion 203, a first arm 204 extending from the central body portion 203, and a second arm 205 extending from an opposite side of the central body portion 203 as the first arm 204. First arm 204 and second arm 205 may curve towards each other, and each of first arm 204 and second arm 205 may be tapered. First arm 204 may include a first tab 206 protruding outward from first arm 204, and first tab 206 may protrude outward towards second arm 205. Second arm 205 may include a second tab 207 protruding outward from second arm 205, and second tab 207 may protrude outward towards first arm 204. Each of first tab 206 and second tab 207 may extend radially inward, e.g. towards axis 198, when actuator 116 is coupled to handle 102. Each of first tab 206 and second tab 207 may be configured to be received by and slide within track 128 of handle 102. Each of first tab 206 and second tab 207 may extend outward from an inner curved surface 211 of actuator 116. A protrusion 202 may extend around an outer curved surface 212 of actuator 116, and protrusion 202 may be configured to facilitate gripping actuator 116 with one or more fingers and/or thumbs. In some aspects, although not shown, actuator 116 may include a plurality of protrusions 202, for example, spaced along a length of actuator 116.

An elastic member 210 may be coupled to actuator 116, may be rectangular in shape, may be ring-shaped and/or an O-ring, or may be any other suitable shape. Elastic member 210 is shown in FIG. 2A with a rectangular, ring-shaped elastic member 210, which may be an “O-ring” or other circular elastic member bent to fit within a slot 236 in actuator 116. In some examples, elastic member 210 may be mounted to slot 236 in inner curved surface 211 of actuator 116. In some examples, elastic member 210 may be insert molded onto actuator 116. Elastic member 210 may facilitate creating a substantially air-tight seal between actuator 116 and vent 121. In some examples, actuator 116 may not include an elastic member 210. In some examples, actuator 116 may not include an elastic member 210, and an elastic member 210 may be coupled around a periphery of vent 121.

In some examples, each tab 206, 207 may include a detent/protrusion 208, 209, respectively. Each protrusion 208, 209 may be configured to be received within recesses 228 of track 128, in order to temporarily prevent movement of actuator 116 through track 128. FIG. 2B illustrates a magnified view of tab 207 and protrusion 209, and FIG. 2C illustrates actuator 116 positioned within track 128 including recesses 228. As shown in FIG. 2C, protrusion 209 is received within a recess 228 of track 128, and may help prevent movement of actuator 116 through track 128. In some examples, the application of suction/aspiration to handle 102 may provide a force pushing or urging actuator 116 outward from handle 102. Thus, a user may release actuator 116 when protrusion 209 is positioned within one recess 228 of track 128, and actuator 116 may remain positioned within recess 228 and not move through track 128 since protrusion 209 is positioned within recess 228. In some examples, protrusion 209 is suctioned towards recess 228 due to suction being applied to opening 156 of handle 102.

In some examples, elastic member 210 may provide a spring-bias to actuator 116 towards a position in which protrusions 208, 209 are positioned within recesses 228 of tracks 128. In this aspect, a user may push actuator 116 towards handle 102 in order to move protrusions 208, 209 out of recesses 228 and then move actuator 116 through track 128. In some examples, the user may release actuator 116, and the spring bias from the elastic member 210 may move protrusions 208, 209 into recesses 228 of tracks 128. A second track 128 may be positioned at an opposite side of handle 102 as the track 128 shown in FIG. 2C, and protrusion 208 may interact with recesses 228 of this second track 128 in the same manner as shown in FIG. 2C. In some examples, recesses 228 may be aligned with markings 177 of handle 102. A user may position actuator 116 around handle 102 and within tracks 128 by pulling each arm 204, 205 away from each other, moving actuator 116 such that inner surface 211 abuts handle 102, and releasing each arm 204, 205 to allow each arm 204, 205 to snap or bend back such that tabs 206, 207 are positioned within tracks 128 of handle 102. Recesses 228 may be evenly spaced or unevenly spaced, and the spacing may correspond to percentages of suction applied to medical device 100.

Actuator 116 is shown in FIG. 1 positioned on a proximal-facing side of handle 102, which may facilitate access to actuator 116 by a user's finger(s) (e.g., a user's thumb) during operation (e.g., while holding handle 102).

Seal 108 may be cylindrical and configured to be positioned around proximal body 106, for example, at tapered protrusion 428. Referring to FIG. 4B showing an exploded view of medical device 100, seal 108 may include a cylindrical outer portion 445 and a cone-shaped inner portion 441 positioned within the cylindrical outer portion 445. A central lumen 443 may extend through seal 108 and may be configured to receive an insertion device 306. Cone-shaped inner portion 441 may be configured to form a seal around an insertion device 306 when insertion device 306 is positioned within lumen 443. Seal 108 may include a tethered plug 446, and tethered plug 446 may be configured to fit within lumen 443 to close off and seal lumen 443, and thus allow sheath 104 to operate (to suction air/fluid/material or deliver air/fluid) through sheath 104 and handle 102, without any insertion device or other component positioned within lumen 443. When tethered plug 446 is positioned within lumen 443, sheath 104 may operate as a suction sheath 104 or a passive drainage sheath 104. In other examples, seal 108 may not include tethered plug 446. Seal 108 may be made of an elastic material, and may form a seal around proximal body 106 when coupled to proximal body 106.

In some examples, medical device 100 includes a biasing member 304. FIG. 3 illustrates a front view of distalmost end 105 of sheath 104 with insertion device 306 and biasing member 304 positioned within sheath 104. As shown in FIG. 3, biasing member 304 may be inserted into sheath 104 such that it occupies one side or portion of sheath 104, and insertion device 306 occupies the other side or portion of sheath 104, creating a channel 307 that is greater in size on the side of the biasing member 304. A radially-inward facing surface 302 of sheath 104 may be spaced from a radially-outward facing surface 308 of insertion device 306, and biasing member 304 may help to form a consistent gap between radially-inward facing surface 302 of sheath 104 and radially-outward facing surface 308 of insertion device 306.

In FIG. 4B, biasing member 304 is shown extending through sheath 104 and proximal body 106, and includes a proximal ring 305 at a proximalmost end of biasing member 304. Proximal ring 305 may be mounted and/or glued to biasing member 304, and may be configured to couple to a countersink (not shown) of proximal body 106. Proximal ring 305 be positioned between proximal body 106 and seal 108, and may help to prevent proximal movement of biasing member 304 during operation. Biasing member 304 may be long enough to extend from a proximal end of medical device 100 to beyond distal end 105 of sheath 104. Biasing member 304 may include a flexible distal portion, and may also include a relatively more rigid portion proximal of the flexible distal portion. Biasing member 304 may be a guidewire, a rod with a flexible distal end, a tube, a retrieval device, and/or an elongate medical device. In some examples, dilator 110 may include a longitudinally extending slot configured to accommodate biasing member 304 and allow both dilator 110 and biasing member 304 to be positioned within sheath 104. In some examples, medical device 100 may not include biasing member 304.

Referring to FIG. 3, insertion device 306 may include a working channel 311 and one or more imaging components 310, such as any combination of one or more cameras and one or more illuminators. Insertion device 306 may be a ureteroscope, a hysteroscope, a bronchoscope, a cystoscope, an endoscope, a duodenoscope, a colonoscope, a catheter, a sheath, a diagnostic or therapeutic tool or device, and/or other types of medical devices. Insertion device 306 may supply fluid aspiration through working channel 311.

During operation, medical device 100 may be coupled to a suction source, such as a vacuum or other suction source. For example, a tube connected to a vacuum source may be coupled to handle 102 at opening 456. A user may insert dilator 110 into sheath 104 and may couple hub 111 to proximal body 106 via one or more latches 127. The user may then position distal portion 172 of sheath 104 at a target site, for example via a guidewire or other means. The user may then remove dilator 110 from sheath 104 and proximal body 106, and insert an insertion device 306 through seal 108 into proximal body 106 and sheath 104. The user may then apply suction to medical device 100 to suction material from a target site within a patient. In some examples, the user may deliver fluid through working channel 311 of insertion device 306. The user may adjust the magnitude of the applied suction by moving actuator 116 along handle 102 to cover vent 121 (to increase the magnitude of the applied suction) or to uncover vent 121 (to decrease the magnitude of the applied suction). By adjusting the magnitude of the suction applied to sheath 104, the user may prevent over-pressurizing a body lumen during a procedure, may prevent injuring the patient due to an excessive suction force, and may decrease procedure time by allowing the user to more efficiently complete a procedure.

Although medical device 100 is described herein for use with an insertion device 306, medical device 100 is not so limited and may be used without insertion device 306 and/or without dilator 110. Medical device 100 may be used for active and/or passive drainage of a patient's body lumen, and in some examples may be operated with tethered plug 446 positioned within seal 108.

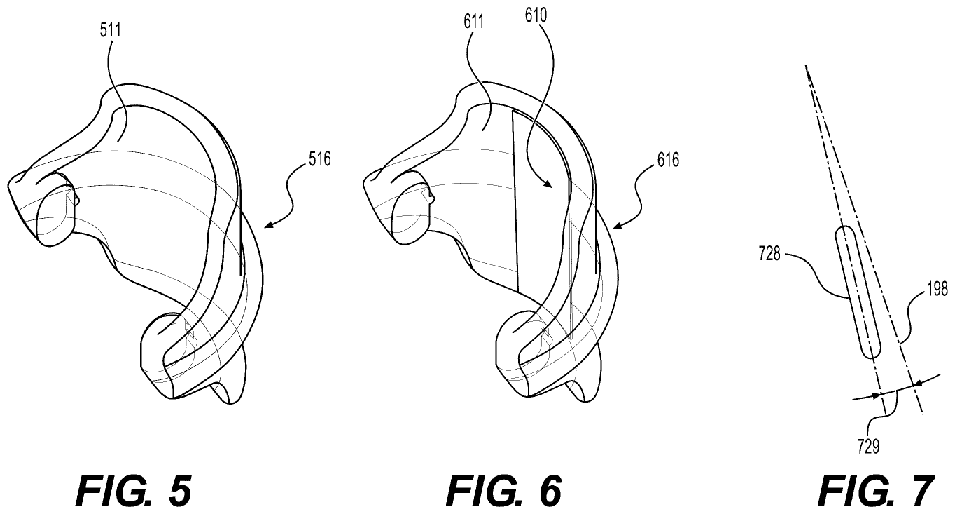

FIG. 5 illustrates an alternative embodiment of actuator 516, which may have any of the features described herein with relation to any other actuator 116 and may be incorporated into any of the medical devices 100 described herein. Actuator 516 does not include elastic member 218 or slot 236. An inner curved surface 511 of actuator 516 may be substantially smooth.

FIG. 6 illustrates an alternative embodiment of actuator 616, which may have any of the features described herein with relation to any other actuator 116, 516 and may be incorporated into any of the medical devices 100 described herein. Actuator 616 includes a rectangular elastic member 610, and rectangular elastic member 610 may be coupled to inner curved surface 611 of actuator 616. In some examples, rectangular elastic member 610 may be coupled within a slot (such as slot 236 described herein above) of inner curved surface 611.

FIG. 7 illustrates an exemplary track 728 and the position of track 728 relative to a central longitudinal axis 198 of handle 102. Other components of medical device 100 have been removed from FIG. 7 for clarity; however, track 728 may be incorporated into handle 102 of medical device 100, for example, by replacing track 128 with track 728. As shown in FIG. 7, a longitudinal axis of track 728 forms an angle 729 (e.g. 15 degrees, 20 degrees, 30 degrees, 45 degrees, or any other suitable angle) with central longitudinal axis 198 of handle 102. By positioning track 728 at angle 729 relative to central longitudinal axis 198 of handle 102, actuator 116 may move closer to handle 102 as actuator 116 is moved proximally through track 728. Accordingly, as actuator 116 is moved through track 728 and moves closer to handle 102, actuator 116 may become tighter against handle 102 to create a tighter seal between handle 102 and actuator 116. Referring to FIG. 4B, if handle 102 incorporates track 728, actuator 116 may move closer to handle 102 as actuator moves between first end 147 and second end 412, which thus creates a tighter seal over vent 121 as actuator 116 is moved proximally from first end 147 to second end 412. In some examples, handle 102 may include two tracks 728 on opposite sides of handle 102.

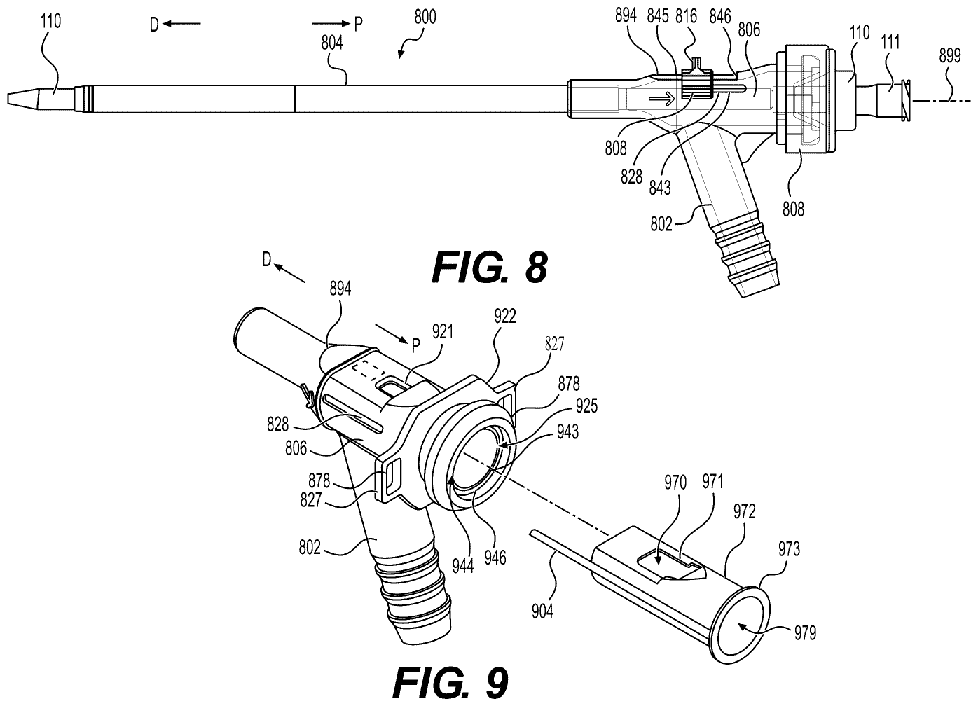

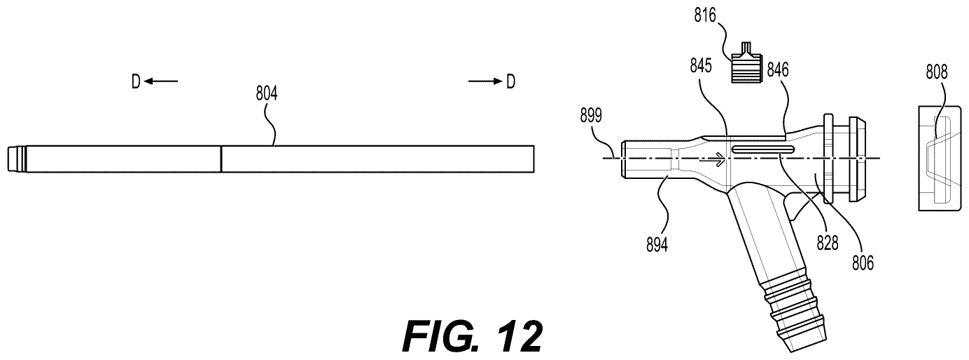

FIG. 8 illustrates a side view of an alternative embodiment of a medical device 800. Medical device 800 may include any of the features described herein in relation to other medical devices 100. Medical device 800 may include a handle 802, a sheath 804, proximal body 806, seal 808, actuator 816, and dilator 110 including dilator hub 111. Medical device 800 may include a central longitudinal axis 899. Handle 802 may not include a vent and actuator 816 may be coupled to proximal body 806. A track 828 may extend longitudinally across proximal body 806, and a portion of actuator 816 may be positioned within track 828. Actuator 816 may be moveable through track 828 from a first end 845, in which actuator 816 completely covers a vent 921 of proximal body 806, to a second end 846, in which actuator 816 does not cover vent 921 of proximal body 806. Proximal body 806 may include a fitting 894 coupled to sheath 804. Actuator 816 may be positioned on an opposite side of proximal body 806 from handle 802. FIG. 12 illustrates an exploded view of medical device 800.

FIG. 9 illustrates proximal body 806 and handle 802, with other components of medical device 800 removed. As shown in FIG. 9, proximal body 806 may include vent 921, a flange 922 including a pair of arms 827 and opening 946 to central lumen 925 extending longitudinally through proximal body 806 from a distalmost end of proximal body 806 to a proximalmost end of proximal body 806. Arms 827 of proximal body 806 may include openings 828 configured to receive one or more portions of dilator 110. For example, latches 127 of dilator 110 may be received within openings 878 and couple to arms 827. Medical device 800 may further include a sleeve 972 configured to be positioned within proximal body 806. Sleeve 972 may be cylindrical and may include a central lumen 979 extending longitudinally through the entirety of sleeve 972. Sleeve 972 may include an opening 971 of a vent 970, and opening 971 may be configured to align with vent 921 when sleeve 972 is positioned within proximal body 806. A biasing member 904 may be coupled to sleeve 972. Biasing member 904 is shown with a distal portion of biasing member 904 removed. Nevertheless, biasing member 904 may have a length configured to extend to a distal end of sheath 804. Biasing member 904 may be positioned outside of lumen 979, and, in some examples, a proximalmost end of biasing member 904 may abut a flange 973 at a proximal end of sleeve 972.

Flange 973 may be configured to abut a portion of proximal body 806 when sleeve 972 is coupled to proximal body 806, to prevent distal movement of sleeve 972 relative to proximal body 806. A countersink 943 of proximal body 806 may be configured to receive flange 973, and countersink 943 may include a notch 944 configured to receive biasing member 904. In some examples, biasing member 904 may be knobbed and bent over notch 944 to couple biasing member 904 to notch 944. Seal 808 may be positioned around both proximal body 806 and sleeve 972 after sleeve 972 is positioned within proximal body 806.

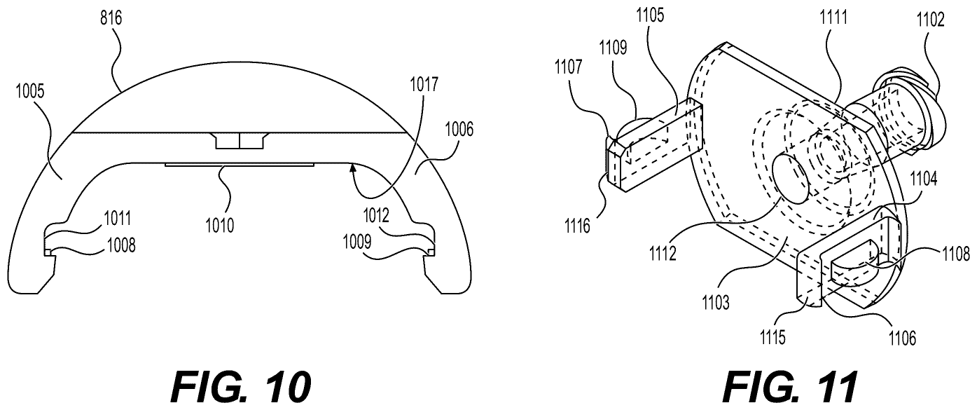

FIG. 10 illustrates a front view of actuator 816 including a first arm 1005 with a recess 1011, a second arm 1006 with a recess 1112, and an elastic member 1010 at an inner surface 1017 of actuator 816. Each recess 1011, 1012 may include a protrusion 1008, 1009, respectively. In some examples, elastic member 1010 may include one or more springs and/or other biasing members to bias actuator 816 away from proximal body 806 when coupled to proximal body 806. When actuator 816 is biased away from proximal body 806, protrusions 1008, 1009 may be biased towards recesses of track 828 similar to actuator 116 and track 128 described herein. In some examples, depressing actuator 816 may cause elastic member 1010 to compress and release protrusions 1008, 1009 from recesses of track 828, and thus allow actuator 816 to be moved proximally or distally within track 828 to cover or uncover vent 921.

FIG. 11 illustrates an exemplary hub 1111 of a dilator 110 described herein. Hub 1111 may include a central body 1103, a central lumen 1112 extending through central body 1103, a proximal end 1102, and a pair of latches 1104, 1105. First latch 1104 may extend distally from central body 1103 and may be positioned on a first edge of central body 1103. Second latch 1105 may extend distally from central body 1103 and may be positioned on a second edge of central body 1103 on an opposite side of central body 1103 from the first edge. Each latch 1104, 1105 may include a tapered distalmost end 1115, 1116, which may facilitate moving each latch 1104, 1105 into openings 878 of proximal body 806. Each distalmost end 1115, 1116 may include a lateral protruding portion 1106, 1107 configured to engage an arm 827 of proximal body 806 to prevent proximal movement of hub 1111 relative to proximal body 806.

A first protrusion 1108 may extend radially outward from first latch 1104, and may be rounded. A second protrusion 1109 may extend radially-outward from second latch 1105 and may be rounded. Each of first protrusion 1108 and second protrusion 1109 may be configured to abut arms 827 of proximal body 806 to help prevent distal movement of hub 1111 relative to proximal body 806 when hub 1111 is coupled to proximal body 806. Each latch 1104, 1105 may be moveable and may be biased towards the position shown in FIG. 11. To couple hub 1111 to proximal body 806, a user may move (e.g., pinch, etc.) latches 1104, 1105 radially inward, push hub 1111 into openings 878 of proximal body 806, and release latches 1104, 1105.

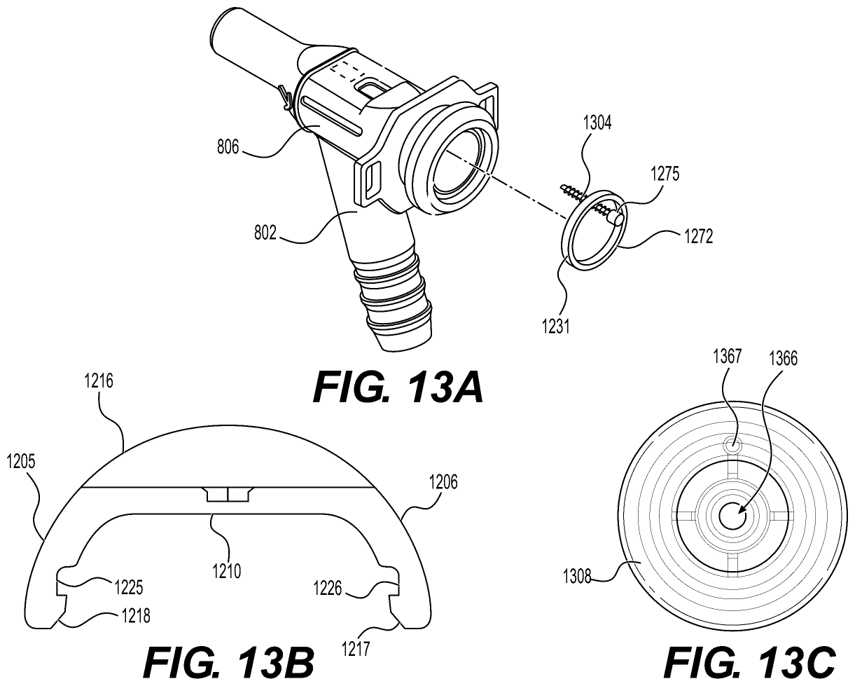

FIG. 13A illustrates an alternative mechanism to couple a biasing member 1304 to proximal body 806. Instead of using sleeve 972, biasing member 1304 may be coupled to a ring member 1231, which is received by proximal body 806. Ring member 1231 may include a protruding portion 1275 extending radially inward relative to a ring portion 1272 of ring member 1231. Protruding portion 1275 may be configured to couple to a proximal end of biasing member 1304. By utilizing ring member 1231 to couple biasing member 1304 to proximal body 806, a notch (to receive biasing member 1304) may not be needed and may not be present on proximal body 806.

FIG. 13B illustrates an alternative embodiment of an actuator 1216 that may be utilized with any of the medical device 100, 800 discussed herein. Actuator 1216 may include a first arm 1205 with a first recess 1225 and first tapered end 1218, and a second arm 1206 with a second recess 1226 and a second tapered end 1217. Actuator 1216 may have any of the features discussed herein in relation to other actuators 116, 816. Actuator 1216 does not include protrusions within recesses 1225, 1226, and does not include an elastic member. Inner surface 1210 may be configured to slide across vent 921 of proximal body 806. Recesses 1225, 1226, may be configured to receive a track that protruded outward from proximal body 806 (e.g., may be a raised track or rail), instead of extending into a recessed track of proximal body 806. Tapered ends 1217, 1218 may help to couple actuator 1216 to proximal body 806, for example, by a user pushing actuator 1216 over tracks of proximal body 806, and tapered ends 1217, 1218 sliding over the tracks of proximal body 806 (e.g., allowing actuator 1216 to be pushed onto proximal body 806). Also, first end 845 and second end 846 of proximal body 806 may be raised to abut actuator 1216 as actuator is either moved distally (to first end 845) or proximally (to second end 846), and thus may limit the proximal and distal movement of actuator 1216 when actuator 1216 is coupled to proximal body 806.

FIG. 13C illustrates an exemplary seal 1308 that may be incorporated into any of the medical devices 100, 800 discussed herein, and seal 1308 may have any of the features of other seals 108, 808 described herein. Seal 1308 may include a central lumen 1366 configured to receive and form a seal around an insertion device 306 or any other insertable medical device. Seal 1308 may also include a smaller lumen 1367 configured to receive and form a seal around a biasing member, such as any of the biasing members 304, 904, 1304 described herein. Smaller lumen 1367 may be self-sealing such that smaller lumen 1367 is sealed (e.g., airtight or closed) when not in use (e.g., when a biasing member or other tool is not positioned within smaller lumen 1367). Smaller lumen 1367 may facilitate exchanging a dilator 110 with an insertion device 306 during a procedure, by allowing a biasing member to remain within the medical device during the removal of a dilator 110 and insertion of an insertion device 306 into the medical device.

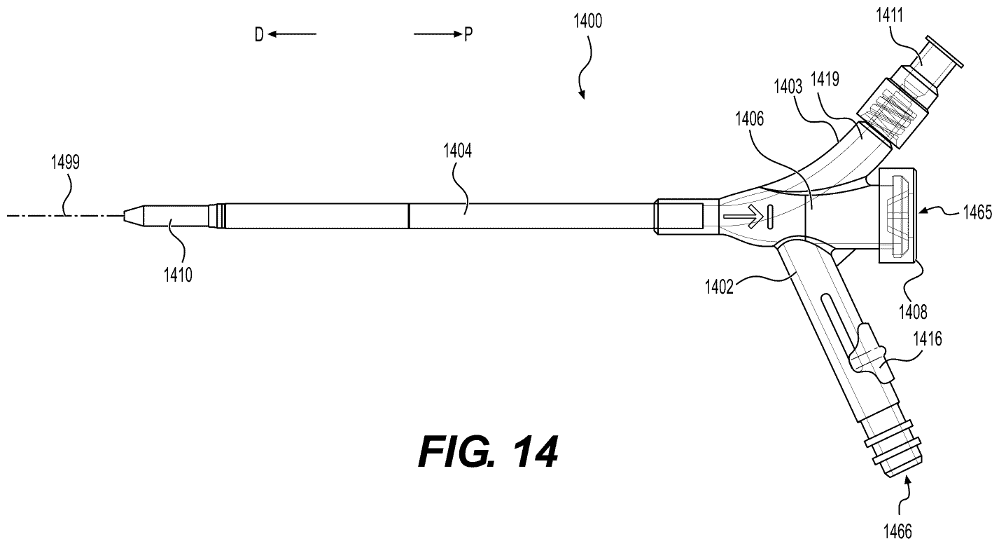

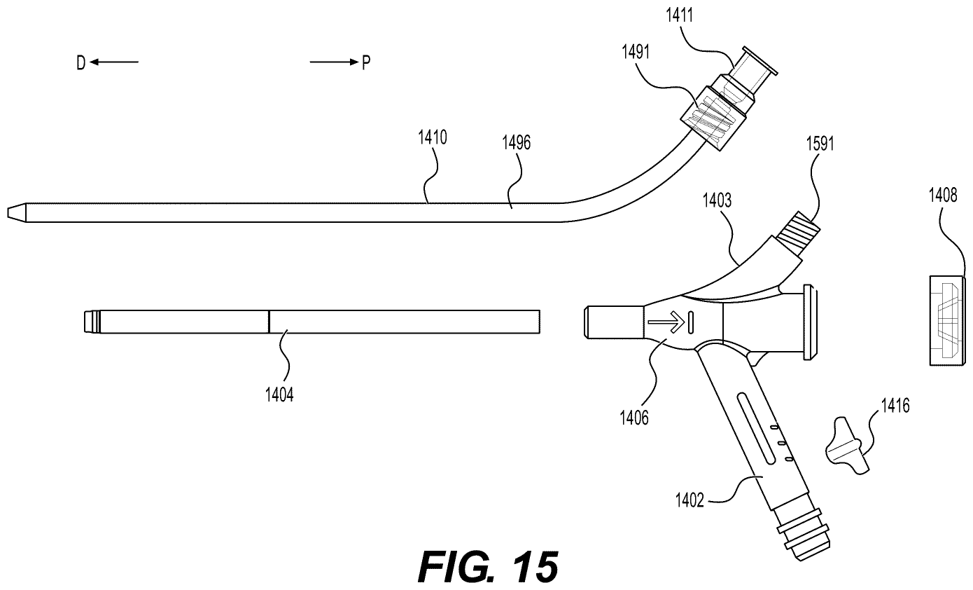

FIG. 14 illustrates an alternative embodiment of medical device 1400, and FIG. 15 illustrates an exploded view of medical device 1400. Medical device 1400 may include any of the features described herein in relation to other medical devices 100, 800. Medical device 1400 may include a handle 1402, a sheath 1404, a proximal body 1406, a seal 1408, an actuator 1416, and a dilator 1410 including a dilator hub 1411. Medical device 1400 may include a central longitudinal axis 1499. Proximal body 1406 may include a first lumen 1465 extending longitudinally through proximal body 1406, and first lumen 1465 may be aligned with central longitudinal axis 1499. A curved arm 1403 of proximal body 1406 may curve away from central longitudinal axis 1499, and curved arm 1403 may include a second lumen 1419 fluidically connected to first lumen 1465. Second lumen 1419 may be configured to receive dilator 1410 and/or other insertable medical tools. Dilator 1410 is shown in FIG. 14 extending through second lumen 1419, into first lumen 1465, and into sheath 1404. A third lumen 1466 may extend through handle 1402 and be fluidically connected to first lumen 1465.

Referring to FIG. 15, dilator hub 1411 may include threads 1491 configured to mate with threads 1591 of curved arm 1403 to couple dilator hub 1411 to curved arm 1403. In some examples, shaft 1496 of dilator 1410 may be removable from dilator hub 1411, and other tools, such as a biasing member or other insertable device, may be coupled to dilator hub 1411 to facilitate coupling to curved arm 1403.

A user may insert insertion device 306 though seal 1408 and proximal body 1406 to a distal end of sheath 1404. Either before or after insertion of insertion device 306 into first lumen 1465, a user may also insert a secondary device (e.g., a biasing member, a snare, laser, etc.) through second lumen 1419 after dilator had been removed from medical device 1400. In some examples, the second device may be coupled to dilator hub 1411 to facilitate coupling to curved arm 1403.

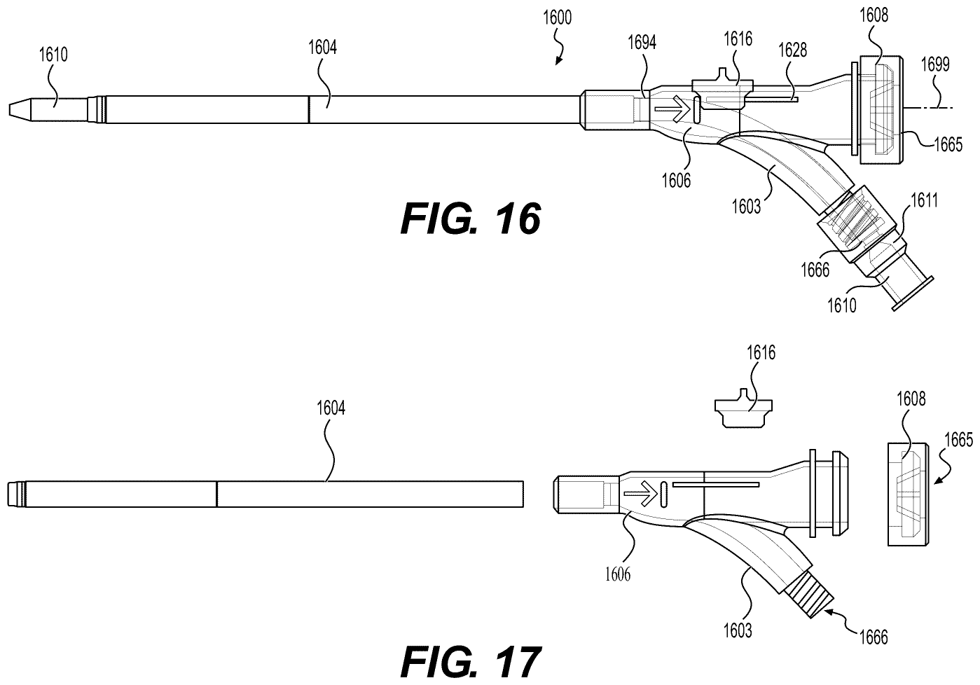

FIG. 16 illustrates an alternative embodiment of medical device 1600, and FIG. 17 illustrates an exploded view of medical device 1600. Medical device 1600 may include any of the features described herein in relation to other medical devices 100, 800, 1400. Medical device 1600 may include a sheath 1604, a proximal body 1606, a seal 1608, an actuator 1616, and a dilator 1610 including dilator hub 1611. Medical device 1600 may include a central longitudinal axis 1699. Proximal body 1606 may include a first lumen 1665 extending longitudinally through proximal body 1606, and first lumen 1665 may be aligned with central longitudinal axis 1699. A curved arm 1603 of proximal body 1606 may curve away from central longitudinal axis 1699, and curved arm 1603 may include a second lumen 1666 fluidically connected to first lumen 1665. Second lumen 1666 may be configured to receive dilator 1610 and/or other insertable medical tools. Dilator 1610 is shown in FIG. 16 extending through second lumen 1666, into first lumen 1665, and into sheath 1604. Dilator 1610 may be identical to dilator 1410 shown in FIG. 15, and may have any of the features described in relation to dilator 1410 or any other dilator discussed herein. Medical device 1600 may not include a handle, and actuator 1616 may be coupled to proximal body 1606, for example, via track 1628 on proximal body 1606. Proximal body 1606 may include a fitting 1694, and fitting 1694 may be coupled to sheath 1604.

During operation, a user may position dilator 1610 within curved arm 1603 and sheath 1604, and then position medical device 1600 at a target site within a patient. The user may then remove dilator 1610 from sheath 1604 and curved arm 1603, and then couple a suction source to curved arm 1603 to apply negative pressure and suction material out of the patient, through sheath 1604 and curved arm 1603. Actuator 1616 may be moved to adjust the magnitude of the applied suction, in the same manner as described herein above in relation to other medical devices 100, 800, 1400.

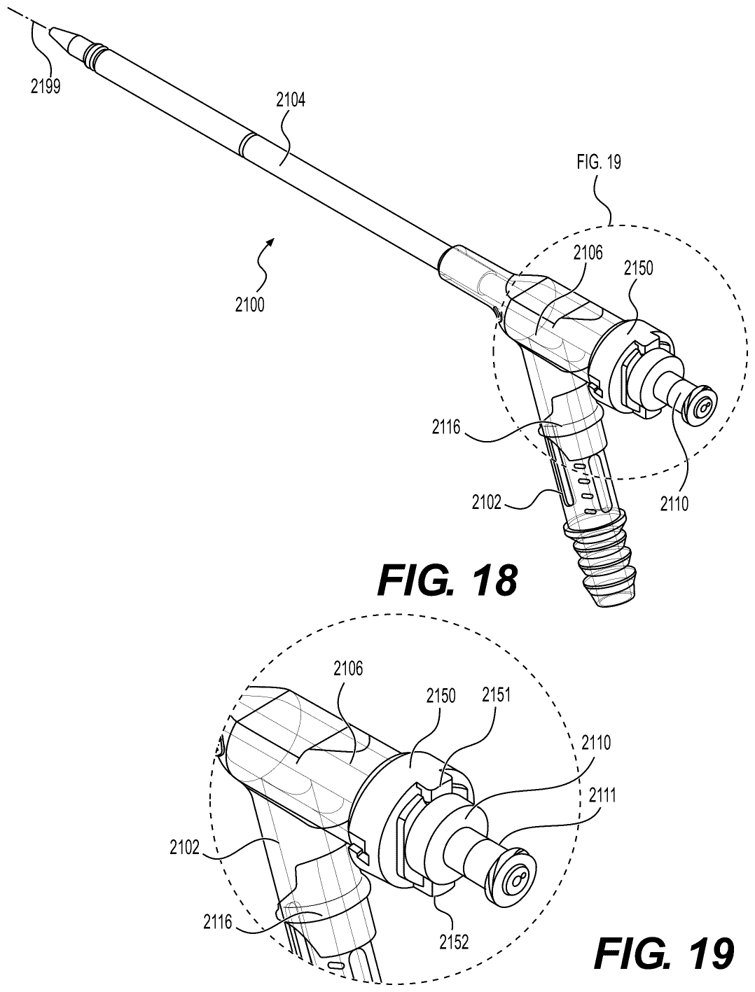

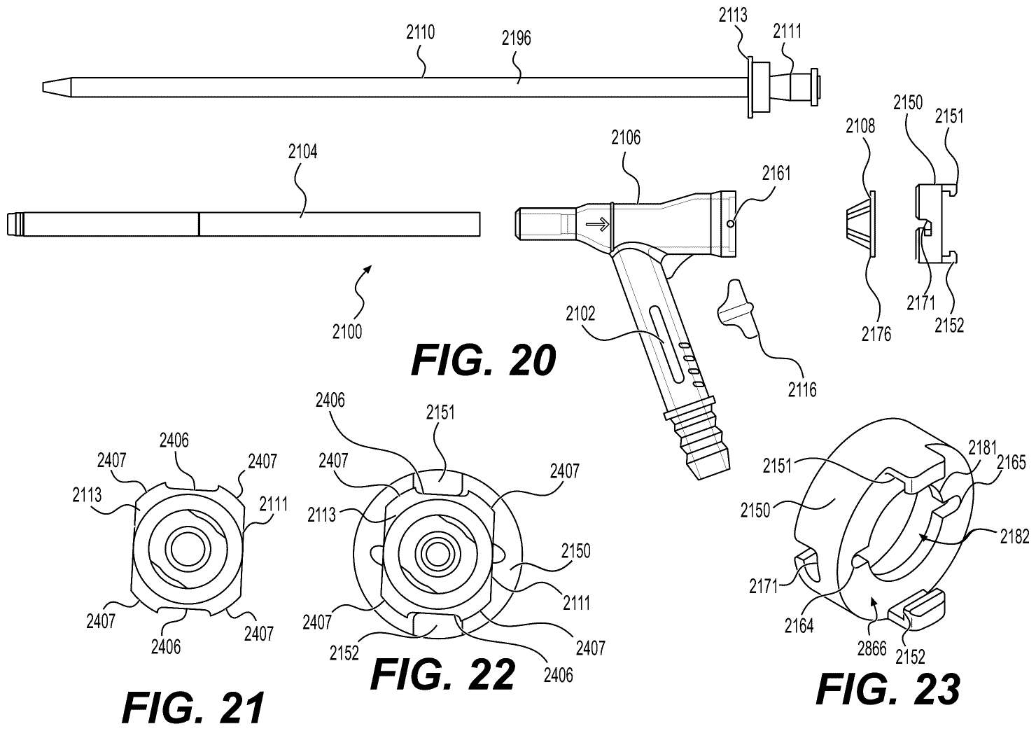

FIG. 18 illustrates an alternative embodiment of medical device 2100. FIG. 19 illustrates a magnified portion of medical device 2100, and FIG. 20 illustrates an exploded view of medical device 2100. Medical device 2100 may include any of the features described herein in relation to other medical devices 100, 800, 1400, 1600. Medical device 2100 may include a handle 2102, a sheath 2104, a proximal body 2106, a seal 2108, an actuator 2116, a dilator 2110 including dilator hub 2111. Medical device 2100 may include a central longitudinal axis 2199. A seal cap 2150 may be coupled to proximal body 2106 at a proximalmost end of proximal body 2106. Seal cap 2150 may include a pair of L-shaped protrusions/arms 2151, 2152 (FIG. 19) extending proximally outward from seal cap 2150. FIG. 19 shows dilator hub 2111 coupled to seal cap 2150, and seal cap 2150 may facilitate coupling dilator hub 2111 to proximal body 2106.

Referring to FIG. 20, proximal body 2106 may include a pair of proximal protrusions 2161 configured to engage seal cap 2150 to couple seal cap 2150 to proximal body 2106. FIG. 23 illustrates seal cap 2150 removed from medical device 2100. As shown in FIG. 23, a pair of L-shaped recesses 2171 of seal cap 2150 may be configured to receive the pair of proximal protrusions 2161 of proximal body 2106. For example, a user may push seal cap 2150 distally onto proximal body 2106, and position proximal protrusions 2161 within recesses 2171, and then turn seal cap 2150 to temporarily fixedly couple seal cap 2150 to proximal body 2106. In other examples, only one L-shaped recess 2171 may be included in seal cap 2150, and proximal body 2106 may include one proximal protrusion 2161 configured to couple seal cap 2150 to proximal body 2106. In other examples, three or more L-shaped recesses 2171 may be included in seal cap 2150, and proximal body 2106 may include three or more proximal protrusions 2161. Seal cap 2150 may include a central lumen 2182 and a central lumen opening 2181 configured to align with an opening 2903 (shown in FIG. 24) of seal 2108. Central lumen opening 2181 may be in a proximal-facing wall 2866 of seal cap 2150. Recesses 2164, 2165 in central lumen opening 2181 may be configured to receive a biasing member or other insertable device, for example if a seal includes a separate lumen for receiving a biasing member or other insertable device separate from dilator shaft 2196.

Seal 2108 may be positioned between seal cap 2150 and proximal body 2106, and seal cap 2150 may facilitate coupling seal 2108 to proximal body 2106. Specifically, seal 2108 may include a circular flange 2176, and flange 2176 may be positioned between seal cap 2150 and proximal body 2106 to help couple seal 2108 to proximal body 2106. FIG. 24 illustrates a perspective view of seal 2108. Seal 2108 may include a tapered portion 2907 forming a funnel shape, and central opening 2903 may be positioned at an apex of the funnel shaped tapered portion 2907. A second lumen 2941 may be positioned on flange 2176, may be self-sealing, and may be configured to receive a biasing member or other insertable device separate from dilator shaft 2196.

Dilator 2110 may include a coupling flange 2113 at a proximal end of dilator hub 2111. FIG. 21 illustrates a distal-facing view of dilator hub 2111. Coupling flange 2113 may include four catches 2407 circumferentially spaced around coupling flange 2113. Recessed slots 2406 may be positioned between catches 2407 of coupling flange 2113. Recessed slots 2406 may be spaced from each other to allow L-shaped arms 2151, 2152 of seal cap 2150 to be positioned within recessed slots 2406 and to freely move proximally and distally through recessed slots. FIG. 22 illustrates L-shaped arms 2151, 2152 positioned within recessed slots 2406. Catches 2407 may be configured to be positioned under L-shaped arms 2151, 2152 of seal cap 2150, to help prevent proximal and distal movement of dilator 2110 relative to seal cap 2150/proximal body 2106.

Specifically, a user may push dilator 2110 distally into medical device 2100 through seal 2108, and push coupling flange 2113 distally beyond a proximalmost end of L-shaped arms 2151, 2152 of seal cap 2150, and then rotate dilator hub 2111 to move one or more catches 2407 in alignment with one or more L-shaped arms 2151, 2152, thus coupling dilator 2110 to proximal body 2106 and preventing proximal and distal movement of dilator 2110 relative to proximal body 2106. By utilizing seal cap 2150 and dilator 2110, a user may more easily couple dilator 2110 to proximal body 2106. Similar to dilator hub 1411, dilator hub 2111 may be removed from dilator shaft 2196, and other medical devices may be coupled to dilator hub 2111 for insertion into medical device 2100, such as a biasing member, retrieval device, laser, or other insertable medical device.

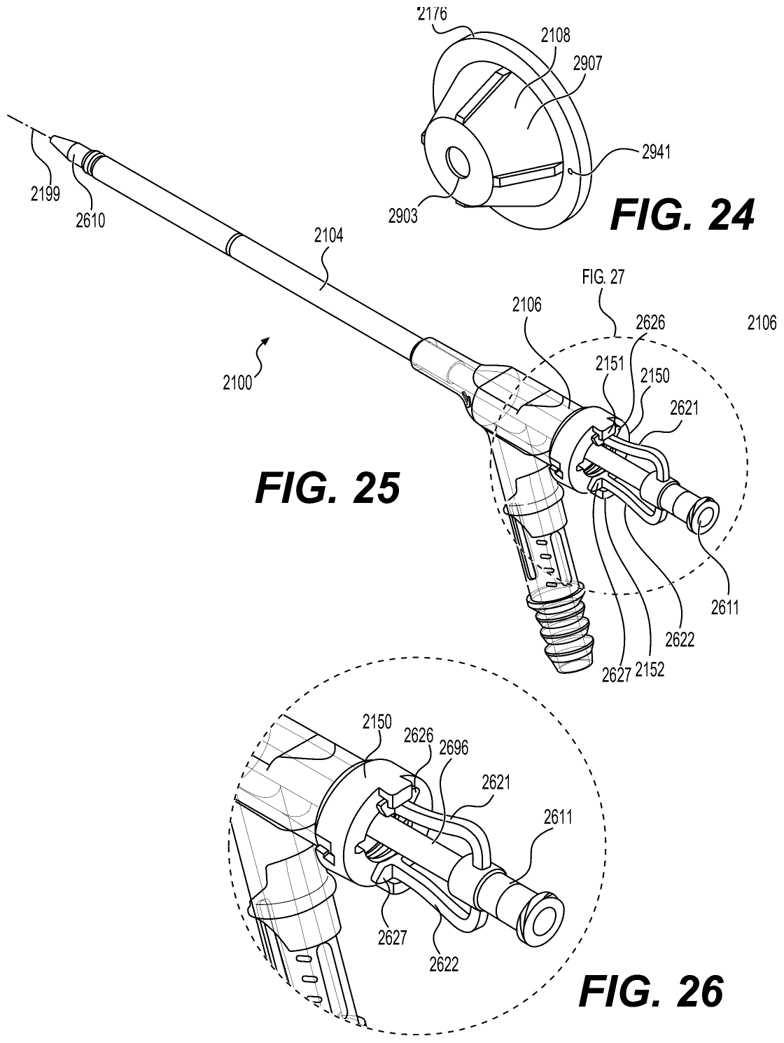

FIG. 25 illustrates medical device 2100 with an alternative embodiment of a dilator 2610 including a dilator hub 2611. FIG. 26 illustrates a magnified portion of medical device 2100 with dilator 2610 coupled to seal cap 2150. Dilator 2610 may include a pair of curved arms 2621, 2622 extending outward and distally from dilator hub 2611. Each curved arm 2621, 2622 may include a flange 2626, 2627, respectively, at its distalmost end. Each flange 2626, 2627 may extend radially outward, relative to central longitudinal axis 2199, from curved arm 2621, 2622, respectively. Each curved arm 2621, 2622 may be biased towards the position shown in FIGS. 23 and 24, and may be flexible such that a user may pinch curved arms 2621, 2622 together to move curved arms 2621, 2622 towards each other, and then release curved arms 2621, 2622 and each curved arm 2621, 2622 will move back to the position shown in FIGS. 23 and 24. Curved arms 2621, 2622 may be configured to couple to seal cap 2150 by engaging L-shaped arms 2151, 2152.

Referring to FIG. 26, flanges 2626, 2627 may be positioned under L-shaped arms 2151, 2152 to couple dilator hub 2611 to seal cap 2150. A user may either pinch curved arms 2621, 2622, push dilator hub 2611 distally beyond a proximal end of each L-shaped arm 2151, 2152 with curved arms 2621, 2622 aligned with L-shaped arms 2151, 2152, and then release curved arms 2621, 2622 to position flanges 2625, 2626 under L-shaped arms 2151, 2152. Alternatively, a user may push dilator hub 2611 distally beyond a proximal end of each L-shaped arm 2151, 2152 with curved arms 2621, 2622 out of alignment with L-shaped arms 2151, and then rotate dilator hub 2611 about axis 2199 to position flanges 2625, 2626 under L-shaped arms 2151, 2152. Similar to dilator hubs 1411, 2111, dilator hub 2611 may be removed from dilator shaft 2696, and other medical devices may be coupled to dilator hub 2611 for insertion into medical device 2100, such as a biasing member, retrieval device, laser, or other insertable medical device.

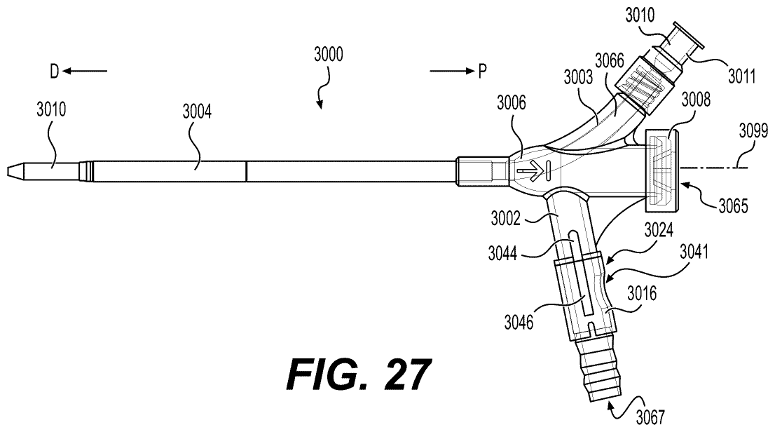

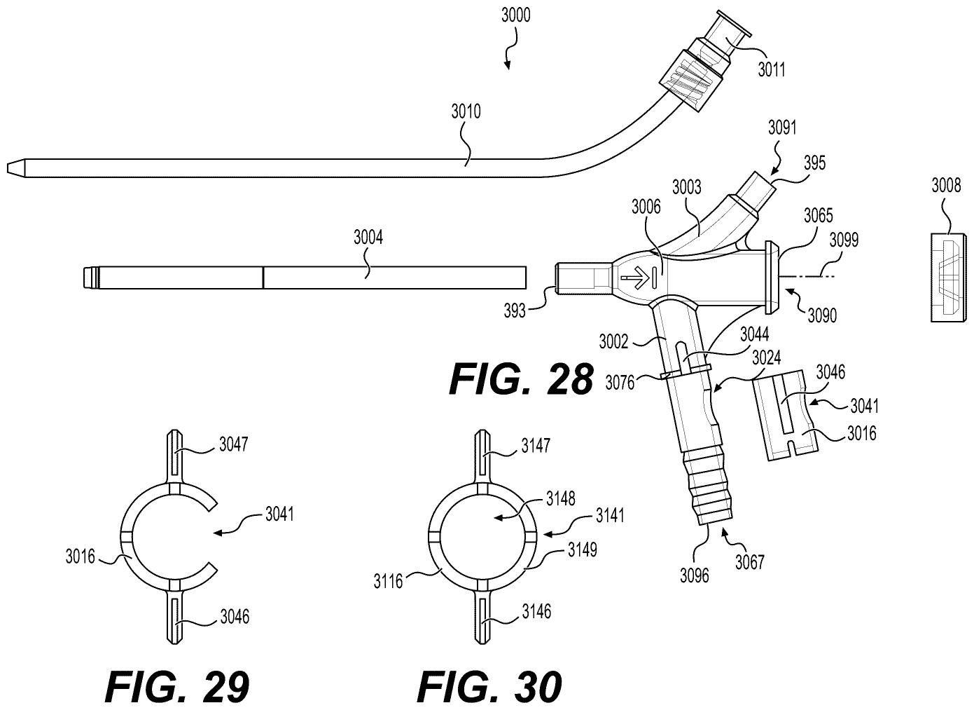

FIG. 27 illustrates an alternative embodiment of medical device 3000, and FIG. 28 illustrates an exploded view of medical device 3000. Medical device 3000 may include any of the features described herein in relation to other medical devices 100, 800, 1400, 1600, 2100. Medical device 3000 may include a handle 3002, a sheath 3004, a proximal body 3006, a seal 3008, an actuator 3016, a dilator 3010 including dilator hub 3011, and a curved arm 3003. Medical device 3000 may include a central longitudinal axis 3099. Seal 3008 may be substantially the same as seals 108, 808, 1408, 1608. Proximal body 3006 may include a first lumen 3065 extending longitudinally through proximal body 3006, and first lumen 3065 may be aligned with central longitudinal axis 3099. Curved arm 3003 of proximal body 3006 may curve away from central longitudinal axis 3099, and curved arm 3003 may include a second lumen 3066 fluidically connected to first lumen 3065. Second lumen 3066 may be configured to receive dilator 3010 and/or other insertable medical tools. Dilator 3010 is shown in FIG. 27 extending through second lumen 3066, into first lumen 3065, and into sheath 3004. A third lumen 3067 may extend through handle 3002 and be fluidically connected to first lumen 3065.

Handle 3002 may include a vent 3024 fluidically connected to lumen 3067, and an alignment marking and/or protrusion 3044. Alignment marking and/or protrusion 3044 may be configured to align with one or more protrusions 3046 of actuator 3016. Actuator 3016 may extend circumferentially around an exterior surface of handle 3002, and may be rotatable relative to handle 3002 about a longitudinal axis of handle 3002. An opening 3041 of actuator 3016 may be moved into and out of alignment with vent 3024, to adjust the magnitude of applied suction via a suction source coupled to handle 3002 (e.g. by adjusting the size of vent 3024). In some examples, opening 3041 may be aligned with vent 3024 when protrusion 3046 is aligned with marking/protrusion 3044 of handle 3002. In other examples, opening 3041 may be entirely out of alignment such that no portion of opening 3041 overlaps with vent 3024 when protrusion 3046 is aligned with marking/protrusion 3044. A user may adjust the magnitude of applied suction by rotating actuator 3016, about a longitudinal axis of handle 3002, relative to handle 3002.

FIG. 29 illustrates an embodiment of actuator 3016 that is C-shaped with protrusions 3046, 3047 at opposite sides of actuator 3016. Protrusions 3046, 3047 may extend radially outward in directions opposite from each other. An opening/gap 3041 may extend along the entire longitudinal length of actuator 3016. In some examples, gap 3041 may have a width that is wider than the width of vent 3024. A user may couple actuator 3016 to handle 3002 by either sliding actuator 3016 onto handle 3002 over proximal end 3096 and to a position abutting flange 3076 of handle 3002. Alternatively, a user may bend actuator 3016 and push handle 3002 through gap 3041 to snap actuator 3016 onto handle 3002 at a position proximal from flange 3076, and then move actuator 3016 to a position in which a distal end of actuator 3016 abuts flange 3076.

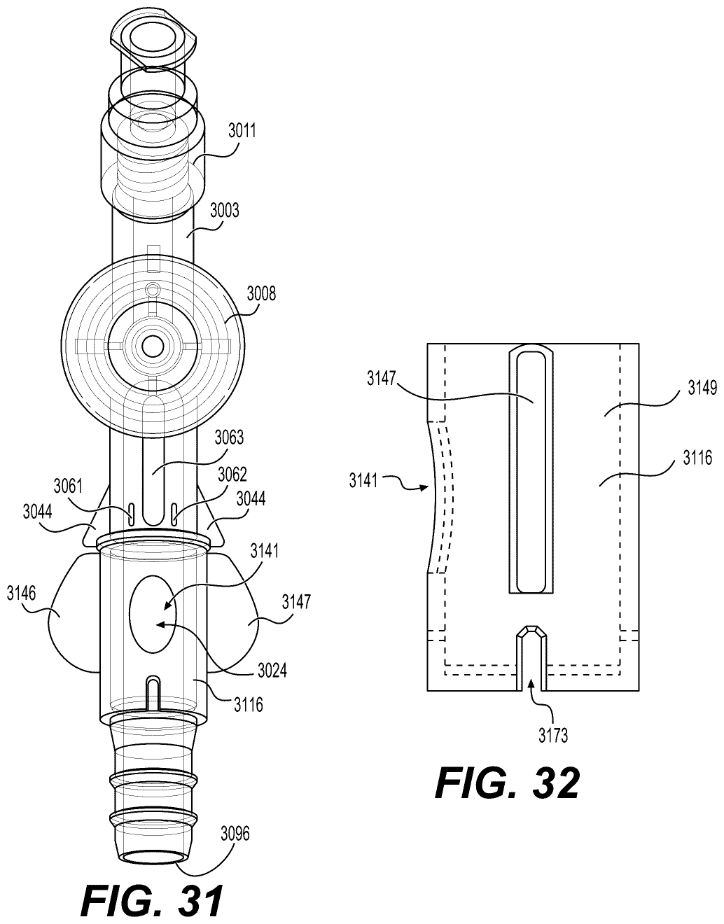

FIG. 30 illustrates an alternative embodiment of an actuator 3116 for medical device 3000. Actuator 3116 may be cylindrical, may have a cylindrical body 3149, a central lumen 3148, and may include protrusions 3146, 3147 at opposite sides of cylindrical body 3149. Protrusions 3146, 3147 may extend radially outward from cylindrical body 3149 in directions opposite from each other. An opening 3141 in cylindrical body 3149 of actuator 3116 may be configured to align with vent 3024 of handle 3002, and may be fluidically connected with central lumen 3148. In some examples, opening 3141 may have a width that is wider than the width of vent 3024. A user may couple actuator 3116 to handle 3002 by sliding actuator 3116 onto handle 3002 over proximal end 3096 and positioning actuator 3116 abutting flange 3076 of handle 3002.

FIG. 31 illustrates a proximal facing view of medical device 3000 with actuator 3116, and shows opening 3141 as an oval-shaped opening 3141 entirely aligned with vent 3024. Protrusions 3044 of handle 3002 are shown aligned with protrusions 3146, 3147 of actuator 3116. Protrusions 3146, 3147 may facilitate a user's grip of actuator 3116 and facilitate rotation of actuator 3116 relative to handle 3002. FIG. 32 illustrates a side view of actuator 3116 including slot 3173. Slot 3173 may be used to fixedly couple actuator 3116 to a position on handle 3002, and/or may be used to snap actuator 3116 onto handle 3002 by allowing expansion of the inner diameter of actuator 3116 for positioning onto handle 3002. In some examples, actuator 3116 does not include slot 3173.

Although actuator 3116 is shown as cylindrical in shape, actuator 3116 may also be hexagonal, octagonal, square shaped, circular, oval shaped, or any other suitable shape. In some examples, either actuator 3116 or actuator 3016 may not include protrusions 3046, 3047, 3146, 3147, and/or either actuator 3016, 3116 may include markings to designate various positions of actuator 3016, 3116 relative to vent 3024. Opening 3141 of actuator 3116 may be circular, elliptical, teardrop shaped, football shaped, multiple circles or multiple of any other shape (e.g., slots, ovals, etc.), and oriented in a horizontal, vertical, angled, or other position relative to a longitudinal axis of handle 3002.

Any of the vents 121, 921, 3024 described herein may have any suitable shape, such as rectangular, oval, circular, square, or any other suitable shape. Vents 121, 921, 3024 may include multiple openings/vents, may be oriented on a single side or multiple sides of a handle 102, 802, 1402, 2102, 3002 or proximal body 106, 806, 1406, 1606, 2106, 3006. Any of the handles 102, 802, 1402, 2102, 3002 and proximal bodies 106, 806, 1406, 1606, 2106, 3006 may include one or more vents 121, 921, 3024.

The medical devices, systems, and methods discussed in this disclosure may facilitate removal of kidney stones or other material from a patient's body, may increase the amount of material that can be removed from a patient during a single procedure, may reduce the procedure time, may reduce the risk of damaging one or more medical devices during a procedure, and may reduce the risk of injury to the patient. For example, the devices in this disclosure may reduce the need for repeated entries into the patient's body with different medical devices during a single procedure.

Any of the medical devices 100, 800, 1400, 1600, 2100, 3000, described herein may be made of any suitable material, such as plastic, PEBAX, or any other suitable material. In any of the above-described examples, medical devices 100, 800, 1400, 1600, 2100, 3000, may include one or more components that are metallic, a polymer, machined, formed, stamped, insert molded, or any combination thereof. Any aspect(s) of any one of the above-described examples of medical device 100, 800, 1400, 1600, 2100, 3000, may be incorporated into any of the other examples of medical devices 100, 800, 1400, 1600, 2100, 3000, described herein.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical device comprising:
a sheath extending from a proximal end to a distal end, wherein the sheath includes a first lumen extending from the proximal end to the distal end;
a proximal body coupled to the proximal end of the sheath and including a second lumen extending longitudinally through the proximal body;
a first body extending radially outward from the proximal body, wherein the first body includes a third lumen extending longitudinally through the first body, wherein the first lumen, the second lumen, and the third lumen are fluidically connected;
a seal positioned at a proximal portion of the proximal body; and
an actuator, wherein actuation of the actuator is configured to adjust a size of a vent of the medical device.

2. The medical device of claim 1, wherein the first body is a handle extending from the proximal body, wherein a longitudinal axis of the handle forms a non-perpendicular angle with a longitudinal axis of the proximal body; and
wherein the handle is configured to be coupled to a suction source.

3. The medical device of claim 2, wherein each of the actuator and the vent is positioned on the handle.

4. The medical device of claim 3, further comprising a dilator positioned within the sheath and the proximal body.

5. The medical device of claim 1, wherein the actuator is C-shaped and includes an elastic member.

6. The medical device of claim 5, wherein at least one protrusion of the actuator is received within a track of the proximal body or the first body.

7. The medical device of claim 6, wherein the track includes a series of recesses configured to receive the at least one protrusion of the actuator.

8. The medical device of claim 6, wherein a longitudinal axis of the track is not parallel with a central longitudinal axis of the first body.

9. The medical device of claim 1, further comprising a biasing member positioned within the sheath and the proximal body.

10. The medical device of claim 9, wherein the biasing member is coupled to a ring member at a proximalmost end of the biasing member.

11. The medical device of claim 9, wherein the biasing member is coupled to a sleeve, and wherein the sleeve is positioned within the second lumen.

12. The medical device of claim 1, wherein the first body is a curved arm extending from a first side of the proximal body, wherein the actuator is positioned on a second side of the proximal body, and wherein the first side is opposite from the second side.

13. The medical device of claim 1, wherein the first body is a curved arm extending from a first side of the proximal body, wherein the actuator is positioned on a handle extending from a second side of the proximal body, and wherein the first side is opposite from the second side.

14. The medical device of claim 1, wherein the actuator is rotatable around a circumference of the second body.

15. The medical device of claim 1, wherein the proximal body includes a pair of arms extending radially outward from a flange of the proximal body, wherein each arm of the pair of arms includes an opening configured to receive a portion of a dilator hub.

16. A medical device comprising:
a sheath extending from a proximal end to a distal end, wherein the sheath includes a first lumen extending from the proximal end to the distal end;
a proximal body coupled to the proximal end of the sheath and including a second lumen extending longitudinally through the proximal body, wherein the first lumen and the second lumen are fluidically connected; and
an actuator, wherein actuation of the actuator is configured to adjust a size of a vent of the medical device.

17. The medical device of claim 16, further comprising a seal at a proximal end of the proximal body, wherein the seal includes a plurality of lumens.

18. The medical device of claim 17, further comprising a seal cap coupled to a proximal end of the proximal body, wherein the seal cap includes a pair of protrusions configured to couple a dilator hub to the seal cap.

19. A medical device comprising:
a sheath extending from a proximal end to a distal end, wherein the sheath includes a first lumen extending from the proximal end to the distal end;
a proximal body coupled to the proximal end of the sheath and including a second lumen extending longitudinally through the proximal body, wherein the first lumen and the second lumen are fluidically connected, and wherein the proximal body include a vent;
an actuator, wherein actuation of the actuator is configured to transition from (i) a first position completely covering the vent to (ii) a second position completely not covering from the vent; and
a seal coupled to the proximal body and configured to receive an insertion device.

20. The medical device of claim 19, further comprising:
a dilator including a dilator hub, wherein the dilator hub includes a pair of arms extending outward from a central body of the dilator; and
a seal cap coupled to the proximal body, wherein the seal cap includes a pair of protrusions configured to engage the pair of arms of the dilator hub to couple the dilator hub to the seal cap.
